(19) United States
(12) Patent Application Publication
(10) Pub. No.: US 2008/0051867 A1
(43) Pub. Date: Feb. 28, 2008

(54) Multiple in vivo implant delivery device

(76) Inventors: Luis A. Davila, Alpharetta, GA (US); Clifford J. Dwyer, Weston, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/511,097

(22) Filed: Aug. 28, 2006

(51) Int. Cl. A61F 2/06

(52) U.S. Cl. 623/1.11

(57) ABSTRACT

A delivery catheter for providing the percutaneous delivery of a plurality of vascular stents. One or more stops are provided in the delivery catheter between each of the plurality of stents. The stops be radiopaque to assist in deploying the stents at desired locations within the vasculature of a patient.

### FIELD OF INVENTION

The present invention relates to the field of medical devices, and more particularly to a device for the delivery of multiple in vivo implant devices.

### DESCRIPTION OF RELATED ART

Vascular disease is a leading cause of premature mortality in developed nations, often presenting as a vascular aneurysm. A vascular aneurysm is a localized dilation of a vessel wall, due to thinning or weakness of the wall structure, or separation between layers of the vessel wall. If untreated, the aneurysm may burst and hemorrhage uncontrollably. Aneurysms are particularly dangerous and prevalent in the aorta, because the aorta supplies blood to all other areas of the body, and because the aorta is subject to particularly high pressures and stresses accordingly. Rupture of an aortic aneurysm is the 15^{th} leading cause of death the United States, afflicting 5% of older men.

It is known to treat vascular aneurysms surgically where blood pressure control medication is unsuccessful at arresting growth of the aneurysm. Surgery often involves the insertion of a vascular stent graft to exclude the aneurysm and carry blood past the dilated portion of the vessel, relieving the pressure on the aneurysm. Other applications for the treatment of vascular disease is the use of a stent to open an occluded vessel, commonly a coronary artery. Such stents may include a coating designed to release a pharmaceutical compound into the bloodstream at a controlled rate.

Moreover, it would be advantageous to design a stent graft that is collapsible to facilitate percutaneous insertion by minimally invasive surgical techniques. Additionally, percutaneous insertion requires the design and development of a delivery apparatus that can effectively position and deploy the vascular stent.

It is often advantageous to provide multiple stents to support a vessel. For example, a vessel may be subject to multiple blockages along its length. Alternately, it often enhances the flexibility of the stent and improves stent placement accuracy to provide plural serial stents rather than a single stent of equivalent length. Therefore, an efficient delivery apparatus for plural in vivo implant devices, such as prosthetic stents, is desired in the art.

### BRIEF SUMMARY OF THE INVENTION

Therefore, in order to overcome these and other deficiencies in the prior art, provided according to the present invention is an apparatus for the percutaneous delivery of plural segmented vascular stents.

### BRIEF DESCRIPTION OF THE DRAWINGS

These and other features, benefits, and advantages of the present invention will be made apparent with reference to the following detailed description, appended claims, and accompanying figures, wherein like reference numerals refer to like structures across the several views, and wherein:

FIG. 1 illustrates a schematic version of a delivery catheter according to a first embodiment described herein.

FIG. 2 illustrates a distal end of the delivery catheter of FIG. 1.

FIG. 2A illustrates a distal portion of the delivery catheter after a first distal stent has been deployed according to the first embodiment described herein.

FIG. 3 illustrates a cross-sectional view of a distal end of a delivery catheter according to the first embodiment described herein.

FIG. 4 illustrates a second embodiment of a delivery catheter according to the description provided herein.

FIG. 5 illustrates a third embodiment of a delivery catheter according to the description provided herein.

FIG. 6 illustrates a fourth embodiment of a delivery catheter according to the description provided herein.

FIG. 7 illustrates a fifth embodiment of a delivery catheter according to the description provided herein.

FIG. 8 illustrates a sixth embodiment of a delivery catheter according to the description provided herein.

FIG. 9 illustrates a seventh embodiment of a delivery catheter according to the description provided herein.

### DETAILED DESCRIPTION OF THE INVENTION

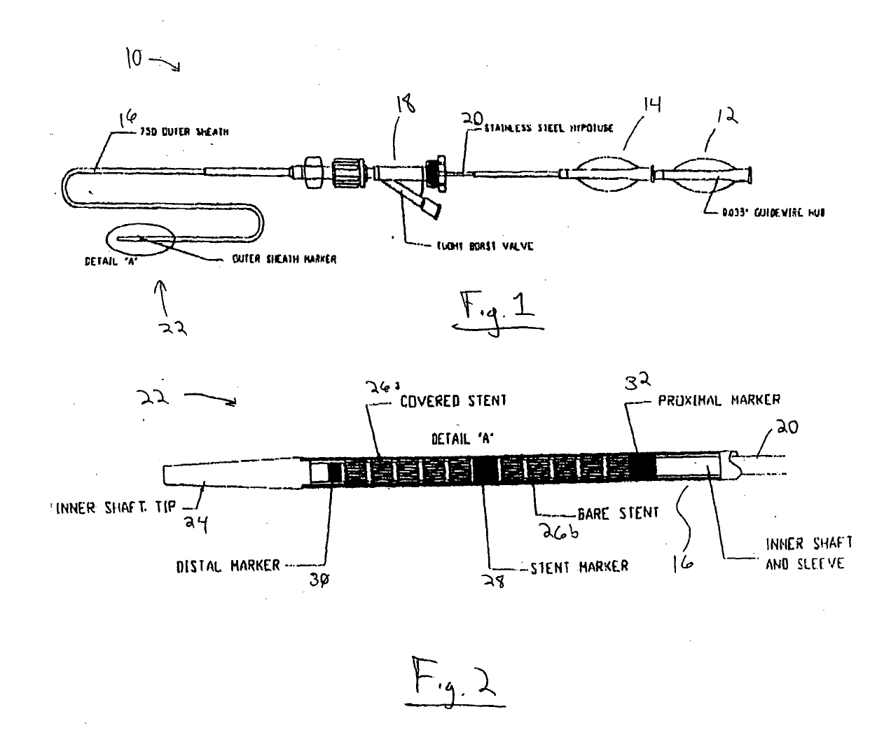

Referring now to FIG. 1, illustrated schematically is a delivery catheter, generally 10, according to the present invention. Delivery catheter 10 has a first handle 12 at a proximal end in communication with a guide wire (not shown) extending axially through the delivery catheter 10. A second handle 14 is in communication with an inner shaft 20 of the delivery catheter 10. An insertion valve 18, preferably a tuohy-borst valve, is in communication with an outer sheath 16 of the delivery catheter 10. Each of the guide wire, the inner shaft 20, and outer sheath 16 are independently axially displaceable relative to one another, by movement of first and second handles 12, 14, with respect to one another and/or to the insertion valve 18.

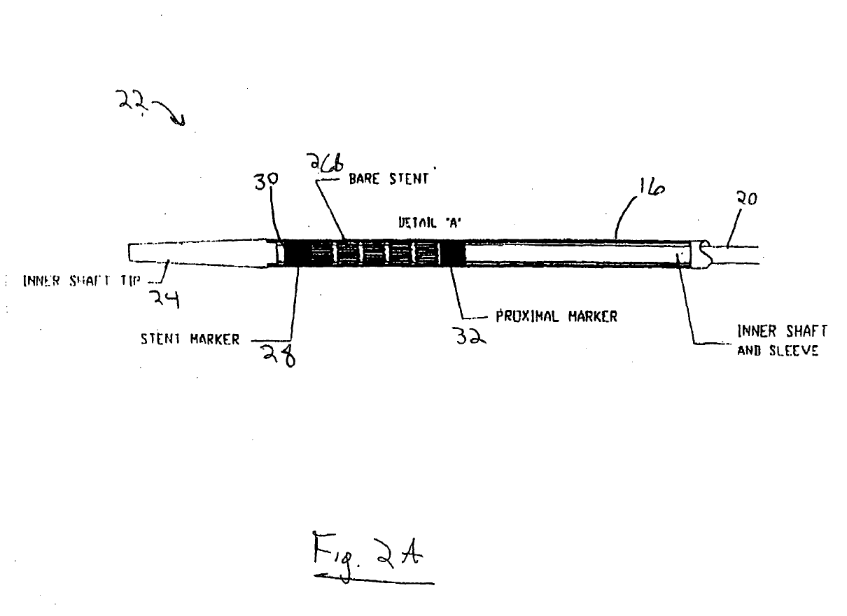

Referring now to FIG. 2, illustrated is a distal end, generally 22, of the delivery catheter 10 for FIG. 1. Inner shaft 20 ends at a distal tip 24. Outer sheath 18 is preferably connected with the distal tip 24, to enable the delivery catheter 10 to rotate as a unit upon insertion and/or to present a continuous outer surface of the delivery catheter 10, thereby reducing resistance to insertion. One or more prosthetic stents 26 are crimped, or reduced in diameter from a fully deployed diameter, onto inner shaft 20 near the distal tip 24. A radiopaque stent marker 28 surrounds the inner shaft 20 between adjacent stents 26a, 26b, thereby radiographically locating, for example by fluoroscopy, the one or more stents 26a, 26b for in vivo delivery and implantation. Additionally, the delivery catheter 10 may include distal radiopaque marker 30 that moves together with the distal tip 24, and proximal radiopaque marker 32 that is secured in axial position along the inner shaft 20.

The stents 26 are preferably self-expanding, and/or comprise a shape memory material, comprising Nitinol or some alloy thereof. In this exemplary embodiment, stent 26a is a covered stent, or one having a coating to, for example, enhance biocompatibility and/or to elute a pharmaceutical compound into the body. By contrast, stent 26b is an uncovered or bare stent. Once positioned as desired by the surgeon, the stents 26a, 26b are deployed individually, by withdrawing the outer sheath 16 from over the stents 26a, 26b, which are then free to expend in the vascular environment.

Preferably, the stents 26a, 26b are deployable individually, and only one stent need be deployed at a time. Referring now to FIG. 2A, after a first distal stent is deployed, in this case covered stent 26a, the distal tip 24 may be withdrawn to meet the outer sheath 16. Stent marker 28 is preferably free-floating over inner shaft 20. Therefore as the distal tip 24 is withdrawn proximally, the stent marker 28 is positioned adjacent a distal end of outer sheath 16, approximately co-located with distal marker 30. Proximal marker 32 having been advanced distally relative to the outer sheath 16 with the deployment of the first distal stent, i.e., covered stent 26a, said proximal marker 32 remains immediately proximal to the bare stant 26b.

Turning then to FIG. 3, illustrated schematically in partial cross-section is a delivery catheter, generally 100, and more specifically a distal end thereof, generally 102. An outer sheath 104 has an axial lumen 106 therethrough, and extends to a distal tip 108. Outer sheath 104 is preferably connected with the distal tip 108, to enable the delivery catheter 100 to rotate as a unit upon insertion and/or to present a continuous outer surface of the delivery catheter 100, thereby reducing resistance to insertion. Inner shaft 110 extents through axial lumen 106 to terminate at the distal tip 108. Preferably, inner shaft 110 has an axial lumen (not shown) running therethrough, to admit a guide wire to assist in inserting the delivery catheter 100.

A plurality of implants 112, for example vascular or other stents, are crimped, i.e., reduced in diameter from a fully deployed diameter to fit within axial lumen 106 for insertion and delivery, to the inner shaft 110. Between each implant 112 is an axial stop 114, which are either free-floating, i.e., axially displaceable over the inner shaft 110 within the axial lumen 106, or secured to the inner shaft 110. A proximal stop 116 is secured to the inner shaft 110 proximally from all implants 112, and limits the axial motion of implants 112 and/or stops 114 between itself and the distal tip 108.

Additionally, the stops 114 may vary in width, either axially, radially, or both, to counteract the stored compressive energy in the delivery catheter 100 during the deployment of successive implants 112. Accordingly, the deployment force required of each implant 112 is more uniform over the course of the procedure, which assists in the more precise and accurate control of deployed position.

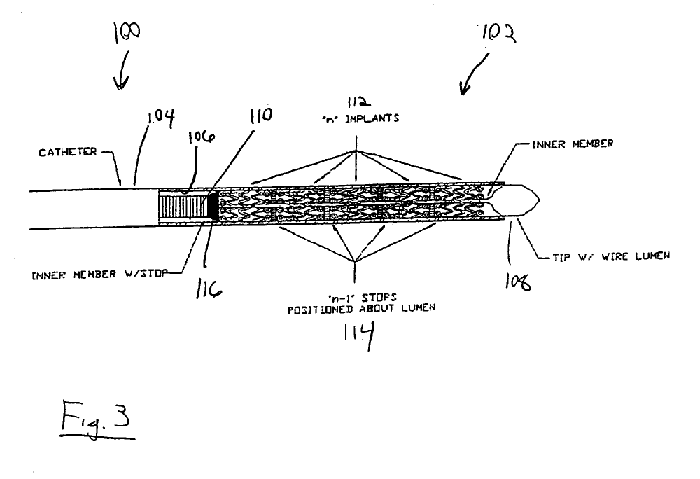

Referring now to FIG. 4, illustrated is an alternate embodiment of a delivery catheter, generally 200. In this embodiment, the stops 214 between each of the implants 112 are radiopaque. The radiopaque stops 214 assist in by radiographically locating, for example by fluoroscopy, the one or more implants 114 for precise control of deployment.

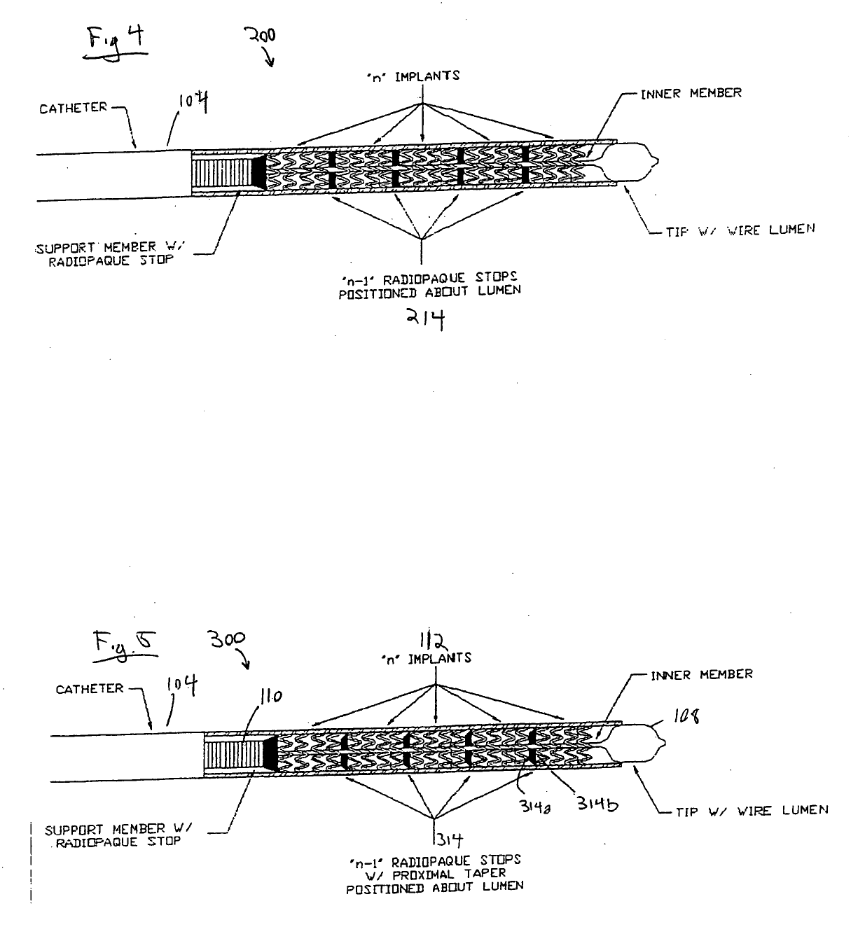

Referring now to FIG. 5, illustrated is an alternate embodiment of a delivery catheter, generally 300. In this embodiment, the stops 314 between each of the implants 112 are proximally tapered, i.e., they are tapered from a narrower diameter on a proximal side 314a to a wider diameter on a distal side 314b. The proximally tapered stops 314 assist may be radiopaque or non-radiopaque.

It is known that self-expanding implants, such as implants 114 have a tendency to ‘jump’, or to move axially from the open end of the outer sheath 104 in the process of expanding to their deployed diameter, as the outer sheath 104 is retracted. The tapered stops 314 assist in the deployment of implants 114 because they allow the implant to pass over the stop 314 with a reduced risk of catching the implant 114 on the stop 314. Therefore, the implant 114 is deployed more consistently without unexpected catching, improving the precision and accuracy of the deployment position.

Referring now to FIG. 6, illustrated is an alternate embodiment of a delivery catheter, generally 400. In this embodiment, implants 412 are provided with integral radiopaque markers 412a, preferably more than one, and preferably distributed over the circumference of the implant 412 at both the proximal and distal ends thereof. In this embodiment, stops 414 are non-radiopaque, to avoid interference with the imaging of the radiopaque markers 412a on the implants 412 themselves.

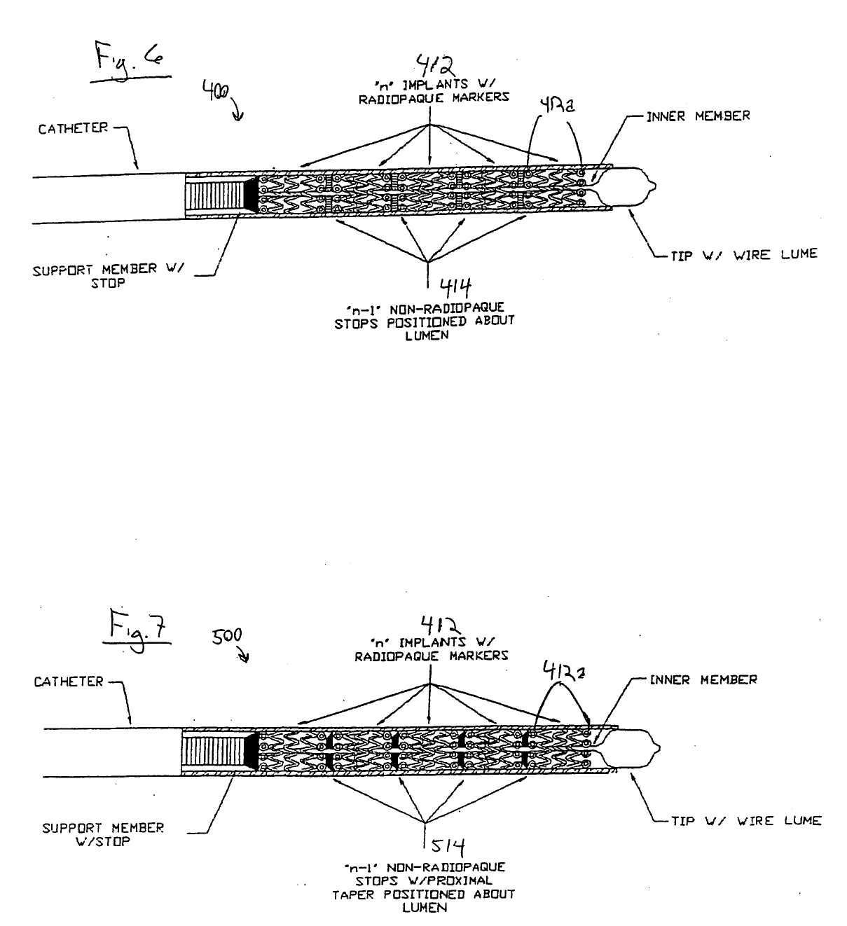

Referring now to FIG. 7, illustrated is an alternate embodiment of a delivery catheter, generally 500. In this embodiment, the stops 514 between each of the implants 112 are proximally tapered, as in the embodiment of FIG. 5. Moreover, the proximally tapered stops 514 are non-radiopaque. Implants 412 have radiopaque markers 412a, as described with reference to FIG. 6.

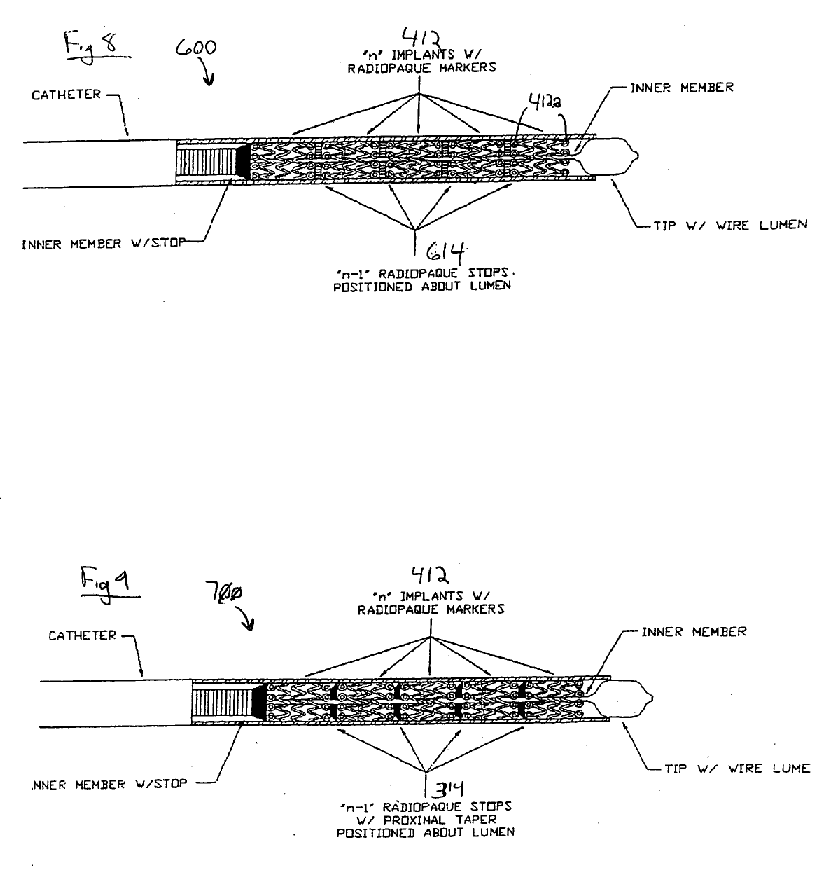

Referring now to FIG. 8, illustrated is an alternate embodiment of a delivery catheter, generally 600. In this embodiment, the stops 614 between each of the implants 112 are of generally constant diameter. Moreover, the constant diameter stops 614 are radiopaque, as described with reference to the embodiment of FIG. 4. Implants 412 have radiopaque markers 412a, as described with reference to FIG. 6.

Referring now to FIG. 9, illustrated is an alternate embodiment of a delivery catheter, generally 700. In this embodiment, the stops 314 between each of the implants 412 are proximally tapered and radiopaque, as described with reference to FIG. 5. Implants 412 have radiopaque markers 412a, as described with reference to FIG. 6.

Accordingly, it will be appreciated from FIGS. 4-9 that the axial stops may be of generally constant diameter or tapered, preferably proximally tapered. Axial stops may be radiopaque or non-radiopaque. The implants themselves may or may not have radiopaque markers. Moreover, any of these features may be used or omitted in any permutation as desired.

The delivery catheter 100 having a central lumen (not shown) for a guide wire will by recognized by those skilled in the art as an over-the-wire type configuration. Alternately, however, the distal tip 108 of the delivery catheter 100 may include an abbreviated passage to accept the guide wire, as part of a so-called rapid-exchange design as is known in the art. Accordingly, the delivery catheter 100 need not be threaded over the entire length of the guide wire, and the guide wire can be shorter. Moreover, using a rapid-exchange design obviates the need for a central lumen to admit the guide wire through all or most of its length. Accordingly, the overall diameter of the delivery catheter can be advantageously reduced. Since the point of connection in the rapid-exchange design is distal of the implants 114, the guide wire would necessarily be outside the implants after deployment, to be subsequently withdrawn.

The present invention has been described herein with reference to certain exemplary or preferred embodiments. These embodiments are offered as merely illustrative, not limiting, of the scope of the present invention. Certain alterations or modifications may be apparent to those skilled in the art in light of instant disclosure without departing from the spirit or scope of the present invention, which is defined solely with reference to the following appended claims.

1. A catheter for the delivery of a plurality of in vivo implant devices, the catheter comprising:
an outer sheath having an axial lumen therethrough;
an inner shaft extending from a proximal end of the catheter to a distal tip through the axial lumen;
a proximal stop secured to the inner shaft and sized to prevent passage through the axial lumen; and
one or more axial stops along the length of the inner shaft between the proximal stop and the distal tip, at least one axial stop axially positioned between two adjacent implant devices.

2. The catheter according to claim 1, wherein at least one of the one or more axial stops are fixed to the inner shaft.

3. The catheter according to claim 1, wherein at least one of the one or more axial stops are axially slidable on the inner shaft.

4. The catheter according to claim 1, wherein at least one of the one ore more axial stops is tapered in diameter along its length.

5. The catheter according to claim 1, wherein at least one of the one or more axial stops is radiopaque.

6. The catheter according to claim 1, wherein at least one of the one or more axial stops comprises a plurality of axial stops, and the axial stops vary in width.

7. The catheter according to claim 6, wherein each of the plurality of axial stops is wider than a distally adjacent axial stop.

8. The catheter according to claim 1, further comprising an axial lumen through at least a part of the inner shaft.

9. The catheter according to claim 1, further comprising a passage at the distal tip for admitting a guide wire, and a rapid exchange mechanism for attaching or detaching the delivery catheter from the guide wire.

10. The catheter according to claim 1, wherein the outer sheath in connected with the distal tip to present a continuous outer surface of the delivery catheter.

11. The catheter according to claim 1, further comprising a plurality of self-expanding in vivo implant devices radially between the inner shaft and the outer sheath, proximally of the proximal stop, and having at least one of said one or more axial stops between any two implant devices.

12. The catheter according to claim 11, wherein one or more of the implant devices comprises one or more radiopaque markers.

13. The catheter according to claim 1, wherein the proximal stop is radiopaque.
